SEQUENCE LISTING (6 sequences)

SEQ ID NO: 1 - DNA, 600 bp, Clostridium difficile
ttacagctttctgattttgataaatttaaaacttaccatctaatactaataacaggttaattttatctaattattatagattctcatactgtgccttattctatctataaatacaatttaagtgtccatattgaaatatttgtattgtaatacagctggatattacttaaatccaattgtttccattataattttatgttaaaataatattacaaaatacatctgtttttcttcataaacgggtgaaattccctatcggcggtaaaagcccgcgagccttatggcataatttggtcatattccaaagccaacagtaaaatctggatggtagaagaaaatagtatatgagtacctttatgtaattttacatgagtaatctatacaaatccttcaactaccgtatttattcatgaaattagacacattcaaggtacctaatatacaggtgctttttttgttgtttattttacaattatatcgtacttataaaatctattaagattggagtgttatcatgaaacaaaaatggatagtattgattatcatctgtattggtgtatttatgtctactcttgatggaagtatactaaatatcgcaaa

SEQ ID NO: 2 - DNA, 116 bp, Clostridium difficile
gtttttcttcataaacgggtgaaattccctatcggcggtaaaagcccgcgagccttatggcataatttggtcatattccaaagccaacagtaaaatctggatggtagaagaaaata

SEQ ID NO: 3 - DNA, 10 bp, Clostridium difficile
atgaaacaaa

SEQ ID NO: 4 - DNA, 19 bp, Clostridium difficile
gtacctaatatacaggtgc

SEQ ID NO: 5 - RNA, 21 nt, Clostridium difficile
agguaccuaauauacaggugc

SEQ ID NO: 6 - RNA, 31 nt, Clostridium difficile
acgggugaaauucccuaucaagguaccuaau

(19) United States
(12) Patent Application Publication
(10) Pub. No.: US 2013/0129695 A1
(43) Pub. Date: May 23, 2013

(54) METHODS FOR TREATING OR INHIBITING INFECTION BY CLOSTRIDIUM DIFFICILE

(71) Applicant: Kenneth Blount, New Haven, CT (US)

(72) Inventor: Kenneth Blount, New Haven, CT (US)

(21) Appl. No.: 13/576,989

(22) PCT Filed: Feb. 4, 2011
(86) PCT No.: PCT/US11/00204

§ 371 Date: Aug. 3, 2012

Related U.S. Application Data
(60) Provisional application No. 61/301,527, filed on Feb. 4, 2010.

(51) Int. Cl. A61K 31/525; A61K 45/06

(52) U.S. Cl. CPC A61K 31/525; A61K 45/06
USPC 424/93.51; 514/251; 514/3.1; 514/236.8; 514/2.9; 514/28; 514/200; 514/192; 436/501

(57) ABSTRACT

The invention provides methods for treating or inhibiting infection by Clostridium difficile in a subject in need of such treatment, comprising administering an effective amount of a compound binding to a CD3299 riboswitch, as well as assays for identifying compounds useful in such treatment, and the use of particular compounds in such treatment.

### CROSS REFERENCE TO RELATED APPLICATIONS

This application claims priority to U.S. Provisional Application No. 61/301,527, filed Feb. 4, 2010, the contents of which are incorporated herein by reference.

### FIELD OF THE INVENTION

This invention relates to compounds and methods for treatment of pathologies caused by Clostridium difficile, to assays to identify compounds useful to treat C. difficile infection, and to a riboswitch of previously unidentified significance and function, which is a target for such treatment.

### BACKGROUND OF THE INVENTION

Riboswitches are regulatory elements found within the 5′-untranslated regions (5′-UTRs) of many bacterial mRNAs. Riboswitches control gene expression in a cis-fashion through their ability to directly bind specific small molecule metabolites. The first domain of the riboswitch, termed the aptamer domain, recognizes and binds the particular ligand, while the second, the expression platform, transduces the binding event into a regulatory switch. The switch includes an RNA element that can adapt to one of two mutually exclusive secondary structures. One of these structures is a signal for gene expression to be “on” and the other conformation turns the gene “off.” Riboswitches are of interest and potential utility as gene switches and as targets for novel antibacterial compounds. See generally Blount, K. and Breaker, R., Riboswitches as Antibacterial Drug Targets, Nature Biotechnology 24, 1558-1564 (2006), the contents of which article are incorporated herein by reference.

Flavin mononucleotide (FMN) riboswitches are found in a wide variety of bacterial species. FMN riboswitches bind to flavin mononucleotide (FMN) and repress the expression of enzymes responsible for riboflavin and FMN biosynthesis. Bioinformatics comparison of all putative FMN riboswitch sequences has led to the proposal of a consensus sequence and secondary structure (Genome Biology 2007, 8:R239) that represents a “consensus FMN riboswitch motif.” This motif is comprised of 6 base-paired helices connected by non-based paired regions. The nucleotide sequences in base-paired and non-base paired regions are generally highly conserved and the consensus FMN riboswitch motif describes the extent of sequence variation at each nucleotide position among all putative FMN riboswitches. A listing of putative FMN riboswitches reported to date can be found in an online database called Rfam (http://rfam.sanger.ac.uk/).

Clostridium difficile is a Gram positive bacteria that may cause diarrhea and other intestinal disease when competing bacteria in the gut are wiped out by antibiotics. Symptoms range from diarrhea to life-threatening inflammation of the colon. Illness from C. difficile most commonly affects older adults in hospitals or in long-term care facilities and typically occurs after use of antibiotic medications. The antibiotics that most often lead to C. difficile infections include fluoroquinolones, cephalosporins, clindamycin and penicillins. Each year, tens of thousands of people in the United States get sick from C. difficile. In recent years, C. difficile infections have become more frequent, more severe and more difficult to treat. Since 2000, more aggressive and dangerous strains of C. difficile have emerged, which are more resistant to antibiotics, produce more deadly toxins than previous strains, and affect otherwise healthy people who are not hospitalized or taking antibiotics.

C. difficile is difficult to treat because it is resistant to many broad spectrum antibiotics, and such antibiotics moreover may cause or maintain the depopulation of healthy intestinal flora, thereby facilitating the C. difficile infection. There is a need for novel approaches to the treatment of pathologies caused by C. difficile, including treatments specific for C. difficile which spare the normal intestinal flora, and treatments which enhance the effectiveness of broad spectrum antibiotics against C. difficile.

### SUMMARY OF THE INVENTION

We have found that a putative FMN riboswitch found in C. difficile, controlling the gene designated CD3299, differs from the consensus motif at nucleotide positions that are normally conserved. To our knowledge, no description or explanation of this motif has been published beyond its appearance in the Rfam database.

Surprisingly, although this riboswitch has been identified based on its sequence as an FMN riboswitch, the cognate ligand for the CD3299 riboswitch seems not be FMN and the molecular recognition of the CD3299 riboswitch is different from other FMN riboswitches. We have determined that the CD3299 riboswitch does not bind to FMN but it does bind to other ligands that we have identified. The CD3299 riboswitch thus has different molecular recognition characteristics from canonical FMN riboswitches. In addition to having a unique sequence, the CD3299 riboswitch resides upstream of and is believed to regulate a putative efflux protein that, if repressed, may impact the antibacterial action of specific ligand classes. Without intending to be bound by theory, it is possible that binding this riboswitch suppresses the expression of this efflux protein, thereby inhibiting the bacterium's ability to excrete toxic chemicals, possibly including antibiotic molecules, so that compounds binding to this riboswitch may be antibacterial as a monotherapy, and/or may enhance the efficacy of other antibiotics.

We have shown that many of the compounds that bind well to the CD3299 riboswitch have improved antibacterial activity toward C. difficile, provided those compounds possess physicochemical characteristics amenable to uptake into the bacteria. Moreover, we have identified compounds that are generally not cytotoxic to mammalian cells at concentrations sufficient to inhibit the bacteria. These compounds are considered to be useful for treatment of pathologies associated with C. difficile, as well as for use as standards in competitive binding assays to identify new compounds binding this target.

Compounds binding the CD3299 riboswitch include compounds as described in our co-pending application, PCT/US 09/04576, the contents of which are incorporated herein by reference.

Accordingly, the invention provides, in a first embodiment, a method of treating pathologies caused by C. difficile by administering an effective amount of a compound, e.g., of Formula IV or Compound 1 or 2, as hereinafter described, which binds to and activates the CD3299 riboswitch, to a patient in need of such treatment.

In a particular embodiment, the C. difficile infection to be treated is resistant to one of more of the following antibiotic classes: fluoroquinolones, cephalosporins, clindamycin and penicillins. In one particular embodiment, the C. difficile infection is resistant to metronidazole (Flagyl) and/or vancomycin (Vancocin) may be prescribed for more severe symptoms. The treatment may further comprise co-administration with one or more additional antibiotics and/or probiotics, e.g., as hereinafter described.

The patient to be treated may be
1. a patient already diagnosed with C. difficile infection, e.g., by real time PCR, cytotoxicity assay for C. difficile toxins, specific toxin ELISA, stool sample, or CT scan for thicken intestinal walls, and/or
2. a patient at elevated risk for C. difficile infection, e.g., selected from patients who have one of more of the following risk factors: (i) are taking or have recently taken broad-spectrum antibiotics, use multiple antibiotics or take antibiotics for a prolonged period, (ii) are 65 years of age or older, (iii) are or have recently been hospitalized, especially for an extended period, (iv) live in a nursing home or long term care facility, (v) have a serious underlying illness or a weakened immune system as a result of a medical condition or treatment (such as pregnancy, chemotherapy, administered immunosuppressive drugs, and/or suffering from systemic lupus erythematosus or any other autoimmune disease), (vi) have had abdominal surgery or a gastrointestinal procedure, (vii) have a colon disease such as inflammatory bowel disease or colorectal cancer, or (viii) have had a previous C. difficile infection.

The patient may, in a further embodiment, be a non-human mammal suffering from suspected C. difficile infection, for example a horse suffering from Colitis-X.

The invention provides the use of compounds in the treatment of pathologies caused by C. difficile, and in the manufacture of medicaments for treatment of pathologies caused by C. difficile. The invention further provides pharmaceutical compositions comprising compounds as hereinbefore described for use in the treatment of pathologies caused by C. difficile infection.

In another embodiment, the invention provides methods of screening or identifying compounds useful for treatment of pathologies caused by C. difficile, comprising measuring the relative binding of a labeled standard to the aptamer domain of the CD3299 riboswitch, in the presence or absence of the test compound.

### DETAILED DESCRIPTION OF THE INVENTION

The 5′UTR and beginning of ORF from CD3299 gene of C. difficile 630, accession number AM180355 is as follows:

SEQ ID NO: 1:
TTACAGCTTTCTGATTTTGATAAATTTAAAACTTACCATCTAATACTAATAACAGGT
TAATTTTATCTAATTATTATAGATTCTCATACTGTGCCTTATTCTATCTATAAATAC
AATTTAAGTGTCCATATTGAAATATTTGTATTGTAATACAGCTGGATATTACTTAAA
TCCAATTGTTTCCATTATAATTTTATGTTAAAATAATATTACAAAATACATCTGTTT
TTCTTCATAAACGGGTGAAATTCCCTATCGGCGGTAAAAGCCCGCGAGCCTTATG
GCATAATTTGGTCATATTCCAAAGCCAACAGTAAAATCTGGATGGTAGAAGAAA
ATAGTATATGAGTACCTTTATGTAATTTTACATGAGTAATCTATACAAATCCTTCAA
CTACCGTATTTATTCATGAAATTAGACACATTCAAG [symbol] TT
TTTTTGTTGTTTATTTTACAATTATATCGTACTTATAAAATCTATTAAGATTGGAGT
GTTATC [symbol] AATGGATAGTATTGATTATCATCTGTATTGGTGTATTTATG
TCTACTCTTGATGGAAGTATACTAAATATCGCAAA

In the above depiction of the sequence, the riboswitch is highlighted in bold, and is

SEQ ID NO: 2
GTTTTTCTTCATAAACGGGTGAAATTCCCTATCGGCGGTAAAAGCCCGCG
AGCCTTATGGCATAATTTGGTCATATTCCAAAGCCAACAGTAAAATCTGG
ATGGTAGAAGAAAATA

The 0RF start site in the above sequence is downstram from the riboswitch and is depicted in italics and is:

SEQ ID NO: 3

[symbol]

The putative terminator hairpin is in bold italics and is:

SEQ ID NO: 4

[symbol]

The hairpin can form a loop having a structure as depicted in Formula 1:

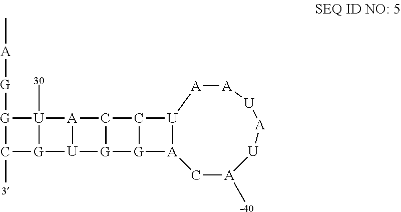

A possible antiterminator has a structure as depicted in Formula 2:

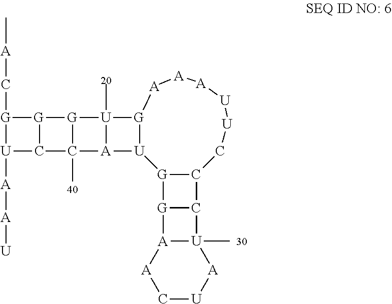

In one embodiment, compounds binding to and activating the CD3299 riboswitch are compounds of Formula IV from PCT Application PCT/US 09/04576:

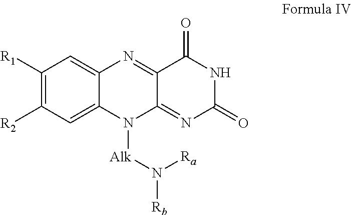

wherein:
(i) Alk is C₁₋₈ alkyl (e.g., ethyl or n-butyl);
(ii) Rₐ and R_{b} are independently H, —C₁₋₄alkyl (e.g., methyl), —(CH₂)₃C(NH₂)(COOH)CHF₂, —(CH₂)₃N(H)C(═NH)NH₂, —(CH₂)₅NH₂, —(CH₂)₂C(H)(OH)COOH, —C(O)(CH₂)₂COOH, —C₁₋₄alkyl-C(O)OR_{S}(e.g., —CH₂CH₂CH₂CH₂C(O)OR₉, —CH₂CH₂CH₂C(O)OR₉, —CH₂CH₂C(O)OR₉ or —CH₂C(O)OR₉, —C(CH₃)(CH₃)C(O)OR₉), —C(O)CH₃, aryl (e.g., phenyl), —C(O)-aryl, aryl-C₁₋₄alkyl (e.g., benzyl, naphtha-1-ylmethyl, naphth-2-ylmethyl, phenylethyl, phenylpropyl, naphtha-1-ylethyl), heteroaryl, heteroaryl-C₁₋₄alkyl (e.g., pyrid-2-ylmethyl, pyrid-3-ylmethyl or quinoxalinyl), wherein said aryl and heteroaryl groups are optionally substituted with one or more groups selected from —C(O)OR₉, —NH₂, —S(O)₂NH₂, —CH₂NH₂, halo (e.g., chloro), C₁₋₄alkoxy (e.g., methoxy), C₁₋₄alkyl (e.g., methyl);
(iii) R₁ is H, C₁₋₈ alkyl (e.g., methyl);
(iv) R₂ is H, halo (e.g., chloro), —O—C₃₋₇cycloalkyl (e.g., —O-cyclopentyl), —N(R₄)(R₅), C₃₋₇cycloalkyl (e.g., cyclopropyl), C₁₋₈alkyl (e.g., methyl or ethyl) or —O—C₁₋₈alkyl wherein the alkyl group is optionally substituted with one or more halo or hydroxyl groups (e.g., trifluoromethyl, —O—CH₂CH₂OH);
(v) R₄ and R₅ are independently H, C₃₋₇cycloalkyl (e.g., cyclopropyl or cyclopentyl), C₁₋₈alkyl (e.g., methyl) wherein said alkyl is optionally substituted with one or more hydroxy groups (e.g., 2,3-dihydroxypropyl, 2,3,4,5,6-pentahydroxyhexyl);
(vi) R₉ is H or C₁₋₄alkyl (e.g., t-butyl, isopropyl, methyl);
(vii) R₁₂ is C₁₋₈alkyl (e.g., methyl, ethyl, t-Butyl) or —OC₁₋₈alkyl (e.g., methoxy, ethoxy, t-butoxy),
   in free, salt or prodrug form.
   In a particular embodiment, the compounds are selected from:

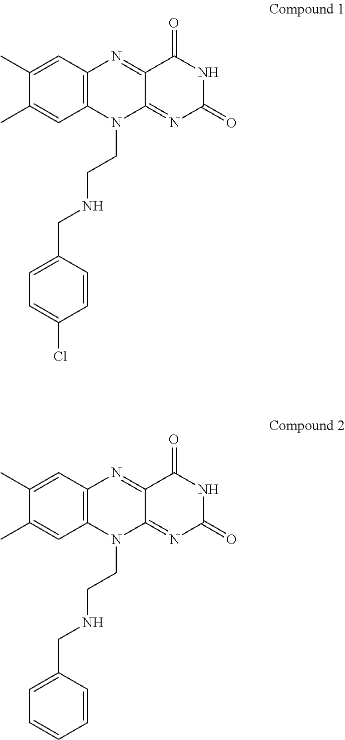 [SMILES: Cc1cc2nc3c(=O)[nH]c(=O)nc-3n(CCNCc3ccc(Cl)cc3)c2cc1C ; Cc1cc2nc3c(=O)[nH]c(=O)nc-3n(CCNCc3ccccc3)c2cc1C]

in free or pharmaceutically acceptable salt form.

The words “treatment” and “treating” are to be understood accordingly as embracing prophylaxis and treatment or amelioration of symptoms of disease as well as treatment of the cause of the disease.

The compounds useful in the methods described herein may be administered orally, parentally (e.g, intravenously), topically, rectally or by other means depending on the nature and location of the infection. Preferably the compounds are administered orally. Dosages employed in practicing the present invention will vary depending, e.g. on the particular disease or condition to be treated, the age and size of the patient, the particular active compound used, the mode of administration, and the therapy desired. For example, in one embodiment, daily oral dosages for a 70 kg human suffering from diarrhea and colitis caused by C. difficile may be from 10-2000 mg. Administration of a therapeutically active amount of the therapeutic compositions is defined as an amount effective, at dosages and for periods of time necessary to achieve the desired result. Dosage regimens may be adjusted to provide the optimum therapeutic response. For example, several divided doses may be administered daily or the dose may be proportionally reduced as indicated by the exigencies of the therapeutic situation.

The compound may administered as monotherapy or in combination with one or more antibiotics, for example in combination with metronidazole (Flagyl®), vancomycin (Vancocin®), linazolid, ramoplanin, and/or fidaxomicin, and/or in combination with one or more antibiotics selected from fluoroquinolones, cephalosporins, clindamycin and penicillins. The patient may receive anti-toxin therapy, for example monoclonal antibodies to C. difficile toxins, or anti-toxoid vaccine. The patient may receive probiotics, such as bacteria and yeast, which help restore a healthy balance to the intestinal tract, e.g., Saccharomyces boulardii (Florastor®), and/or be undergoing fecal bacteriotherapy.

Pharmaceutical compositions comprising compounds as described herein may be prepared using conventional diluents or excipients and techniques known in the galenic art. Thus oral dosage forms may include tablets, capsules, solutions, suspensions and the like. The term “pharmaceutically acceptable carrier” as used herein is intended to include diluents such as saline and aqueous buffer solutions, as well as solid carriers such as microcrystalline cellulose, hydroxypropylmethyl cellulose, or lactose.

### EXAMPLES

### Example 1

### Binding of Compounds to Riboswitch

An in-line probing assay, as described in Regulski and Breaker, “In-line probing analysis of riboswitches”, (2008), Methods in Molecular Biology, Vol 419, pp 53-67, the contents of which are incorporated by reference, is used to estimate the dissociation binding constants for the interaction of each of the ligands described herein with a CD3299 riboswitch amplified from Clostridium difficile. Precursor mRNA leader molecules are prepared by in vitro transcription from templates generated by PCR and [5′-³²P]-labeling using methods described previously (Regulski and Breaker, In-line probing analysis of riboswitches (2008), Methods in Molecular Biology Vol 419, pp 53-67). Approximately 5 nM of labeled RNA precursor is incubated for 41 hours at 25° C. in 20 mM MgCl₂, 50 mM Tris/HCl (pH 8.3 at 25° C.) in the presence or absence of a fixed concentration of each ligand. Binding to the CD3299 riboswitches are measured 100 [symbol] M. In-line cleavage products are separated on 10% polyacrylamide gel electrophoresis (PAGE), and the resulting gel is visualized using a Molecular Dynamics Phosphorimager. The location of products bands corresponding to cleavage are identified by comparison to a partial digest of the RNA with RNase T1 (G-specific cleavage) or alkali (nonspecific cleavage).

In-line probing exploits the natural ability of RNA to self-cleave at elevated pH and metal ion concentrations (pH≈8.3, 25 mM MgCl₂) in a conformation-dependent manner. For self-cleavage to occur, the 2′-hydroxyl of the ribose must be “in-line” with the phosphate-oxygen bond of the internucleotide linkage, facilitating a S_{N}2P nucleophilic transesterification and strand cleavage. Typically, single-stranded regions of the Riboswitch are dynamic in the absence of an active ligand, and the internucleotide linkages in these regions can frequently access the required in-line conformation. Binding of an active ligand to the riboswitch generally reduces the dynamics of these regions, thereby reducing the accessibility to the in-line conformation, resulting in fewer in-line cleavage events within those regions. These ligand-dependent changes in RNA cleavage can be readily detected by denaturing gel electrophoresis. The relative binding affinity of each ligand is expressed as Iₘₐₓ, wherein Iₘₐₓ represents the percent inhibition of in-line cleavage at selected internucleotide ligands in the presence of a fixed ligand concentration (100 [symbol] M for the CD3299 riboswitch) normalized to the percent inhibition in the absence of ligand and the percent inhibition in the presence of a saturation concentration of a control ligand. 100 [symbol] M of Compound A (which is a compound identified as having high affinity to the CD3299 riboswitch) is used as a control ligand for estimating binding to the CD3299.

The experiments show that Compounds 1 and 2 have a binding affinity to the CD3299 switch with an Iₘₐₓ value of 45-90 [symbol] compared to the control at 100 [symbol] M.

### Example B

### Minimum Inhibitory Concentration (MIC) Assay

The MIC assays are carried out in a final volume of 100 μL in 96-well clear round-bottom plates according to methods established by the Clinical Laboratory Standards Institute (CLSI). Briefly, test compound suspended in 100% DMSO (or another suitable solubilizing buffer) is added to an aliquot of media appropriate for a given pathogen to a total volume of 50 μL. This solution is serially diluted by 2-fold into successive tubes of the same media to give a range of test compound concentrations appropriate to the assay. To each dilution of test compound in media is added 50 [symbol] L of a bacterial suspension from an overnight culture growth in media appropriate to a given pathogen. Final bacterial inoculum is approximately 10⁵-10⁶ CFU/well. After growth for 18-24 hours at 37° C., the MIC is defined as the lowest concentration of antimicrobial agent that completely inhibits growth of the organism as detected by the unaided eye, relative to control for bacterial growth in the absence of added antibiotic. Ciprofloxacin is used as an antibiotic-positive control in each screening assay. Each of the bacterial cultures that are available from the American Type Culture Collection (ATCC, www.atcc.org) is identified by its ATCC number.

The experiments show that Compounds 1 and 2 have a minimum inhibitory concentration (MIC) of 64 μg/mL or less against C. difficile strains ATCC 700057 (MMX 4381) and MMX3581 (clinical).

All references indicated herein are incorporated by reference for any patent application in the United States.

1. A method of treating or inhibiting infection by Clostridium difficile in a subject in need of such treatment, comprising administering an effective amount of a compound binding to a CD3299 riboswitch.

2. The method of claim 1 wherein the subject is a human or equine.

3. The method of claim 1 wherein the symptoms of infection include diarrhea or colitis.

4. The method according to claim 1 further comprising administration of one or more additional antibiotics, and/or administration of one or more probiotics.

5. The method of claim 1 wherein the compound is a compound of Formula IV, Compound 1, or Compound 2, in free or pharmaceutically acceptable salt form.

6. (canceled)

7. A method of identifying a compound useful in treating C. difficile infection comprising measuring the binding of a reference ligand to the aptamer domain of the CD3299 riboswitch, in the presence or absence of a test compound.

8. The method of claim 7 wherein the reference ligand is Compound 1 or Compound 2.

9. The method according to claim 4, wherein the antibiotic is selected from the group consisting of: metronidazole, vancomycin, linazolid, ramoplanin, fidaxomicin, fluoroquinolones, cephalosporins, clindamycin and penicillins.

10. The method according to claim 4, wherein the probiotic is Saccharomyces boulardii.

11. The method according to claim 1, wherein the C. difficile infection to be treated is resistant to an antibody class selected from the group consisting of: fluoroquinolones, cephalosporins, clindamycin and penicillins.

12. The method according to claim 1, wherein the C. difficile infection is resistant to metronidazole or vancomycin.

13. The method according to claim 1, wherein the subject is a patient diagnosed with a C. difficile infection by a diagnostic tool selected from the group consisting of: real time PCR, cytotoxicity assay for C. difficile toxins, specific toxin ELISA, stool sample, and CT scan for thickened intestinal walls.

14. The method according to claim 5, wherein the subject is a patient diagnosed with a C. difficile infection by a diagnostic tool selected from the group consisting of: real time PCR, cytotoxicity assay for C. difficile toxins, specific toxin ELISA, stool sample, and CT scan for thickened intestinal walls.

15. The method according to claim 1, wherein the subject is a patient with an elevated risk for a C. difficile infection, wherein said patient has one or more of the risk factors selected from the group consisting of:
taking or having recently taken broad-spectrum antibiotics, use of multiple antibiotics, or has taken antibiotics for a prolonged period;
is 65 years of age or older;
is presently hospitalized or has been recently hospitalized for an extended period;
lives in a nursing home or long term care facility;
has a serious underlying illness or weakened immune system which is a result of a medical condition or treatment;
has had abdominal surgery or a gastrointestinal procedure;
has a colon disease such as inflammatory bowel disease or colorectal cancer; and
has had a previous C. difficile infection.

16. The method according to claim 5, wherein the subject is a patient with an elevated risk for a C. difficile infection, wherein said patient has one or more of the risk factors selected from the group consisting of:
taking or having recently taken broad-spectrum antibiotics, use of multiple antibiotics, or has taken antibiotics for a prolonged period;
is 65 years of age or older;
is presently hospitalized or has been recently hospitalized for an extended period;
lives in a nursing home or long term care facility;
has a serious underlying illness or weakened immune system which is a result of a medical condition or treatment;
has had abdominal surgery or a gastrointestinal procedure;
has a colon disease such as inflammatory bowel disease or colorectal cancer; and
has had a previous C. difficile infection.
